(19) United States
(12) Patent Application Publication
(10) Pub. No.: US 2025/0019669 A1
(43) Pub. Date: Jan. 16, 2025

(54) CELL LINES WITH IMPROVED AAV PRODUCTION CAPACITY

(71) Applicant: THE CHILDREN'S HOSPITAL OF PHILADELPHIA, Philadelphia, PA (US)

(72) Inventors: Beverly L. DAVIDSON, Philadelphia, PA (US); Jonathan F. LANG, Philadelphia, PA (US); Ophir H. SHALEM, Philadelphia, PA (US); Emily O'DRISCOLL, Philadelphia, PA (US); Sakshi ARORA, Philadelphia, PA (US)

(73) Assignee: THE CHILDREN'S HOSPITAL OF PHILADELPHIA, Philadelphia, PA (US)

(21) Appl. No.: 18/713,466

(22) PCT Filed: Dec. 2, 2022
(86) PCT No.: PCT/US2022/080855

§ 371 Date: May 24, 2024

Related U.S. Application Data
(60) Provisional application No. 63/285,381, filed on Dec. 2, 2021.

(51) Int. Cl. C12N 7/00; C12N 5/071; C12N 5/073

(52) U.S. Cl. CPC C12N 7/00; C12N 5/0603; C12N 5/0686; C12N 2510/02; C12N 2750/14143; C12N 2750/14152

(57) ABSTRACT

Provided herein are systems for increasing AAV particle production. These systems comprise producer cell lines adapted for the production of AAV particles, as well as methods of producing AAV particles using said producer cell lines. Also provided are AAV particles produced by said production systems, producer cell lines and methods.

### REFERENCE TO RELATED APPLICATIONS

The present application claims the priority benefit of U.S. provisional application No. 63/285,381, filed Dec. 2, 2021, the entire contents of which are incorporated herein by reference.

### BACKGROUND

### 1. Field

The present disclosure relates generally to the fields of medicine and virology. More particularly, it concerns genetically engineered producer cell lines for improved production of adeno-associated viral particles.

### 2. Description of Related Art

AAV-mediated gene therapy is a growing field with the potential to treat numerous life-threatening human diseases. A major limitation for any AAV-mediated gene therapy is the difficulty in scaling AAV vector production for commercial use. AAV vectors are typically produced by triply transfecting HEK 293 cells with two helper AAV plasmids and the gene of interest followed by harvesting AAV particles from these cells. Compositions and methods for improving AAV production capacity are needed.

### SUMMARY

In one embodiment, provided herein are genetically engineered producer cell lines in which the expression of at least one of TMED10, MON2, TMED2, HS2ST1, C3orf58, SPPL3, SURF4, LSM5, ARF1, and PI4 KB is reduced as compared to a control cell line, and/or in which the expression of at least one of B4GALT7, B3GAT3, OAF, EXT2, COMMD3, SLC35D1, B3GALT6, SDC1, NDST1, RAC1, CSK, GLCE, PDCL, FAM20B, TM4SF5, DGAT2, POMT1, YY1, and DPF2 is increased as compared to a control cell line. In some aspects, the producer cell line is a human embryonic kidney 293T (HEK293T) cell line.

In some aspects, the expression of at least one of TMED10, MON2, TMED2, HS2ST1, C3orf58, SPPL3, SURF4, LSM5, ARF1, and PI4 KB is reduced as compared to a control cell line. In some aspects, the expression of TMED10, MON2, and/or TMED2 are reduced as compared to a control cell line. The expression of at least one of TMED10, MON2, TMED2, HS2ST1, C3orf58, SPPL3, SURF4, LSM5, ARF1, and PI4 KB may be reduced using CRISPR genome editing, a double stranded RNA (dsRNA), a small interfering RNA (siRNA), a small hairpin RNA (shRNA), a micro RNA or an antisense RNA.

In some aspects, at least one of TMED10, MON2, TMED2, HS2ST1, C3orf58, SPPL3, SURF4, LSM5, ARF1, and PI4 KB is knocked out. In some aspects, the producer cell line does not express TMED10, MON2, and/or TMED2.

In some aspects, the expression of one or more of the additional genes listed in FIGS. 5A and 5B is modulated in the producer cell line. In some aspects, the producer cell line comprises a viral vector.

In some aspects, the producer cell line is stably or transiently transfected with a first heterologous nucleic acid sequence, and optionally stably or transiently transfected with a second heterologous nucleic acid sequence. In some aspects, the first heterologous nucleic acid sequence encodes a therapeutic protein or polynucleotide sequence and the optional second heterologous nucleic acid sequence encodes a therapeutic protein or polynucleotide sequence. In some aspects, the therapeutic protein or polynucleotide sequence encoded by the first heterologous nucleic acid sequence and the therapeutic protein or polynucleotide sequence encoded by the optional second heterologous nucleic acid sequence are the same or are different. In some aspects, the therapeutic polynucleotide sequence is an inhibitory nucleic acid sequence, and wherein the inhibitory nucleic acid sequence comprises a small or short hairpin (sh)RNA, microRNA (miRNA), small or short interfering (si) RNA, trans-splicing RNA, or antisense RNA. In some aspects, the first heterologous nucleic acid sequence is comprised within a first viral vector, and the optional second heterologous nucleic acid sequence is comprised within a second viral vector. In some aspects, the first and/or second viral vector comprises an AAV vector genome. In some aspects, the AAV vector genome(s) comprises one or two AAV ITRs that flank the 5′ and/or 3′ ends of the heterologous nucleic acid sequence.

In some aspects, the producer cell line further comprises AAV rep and/or cap encoding sequences. In some aspects, the producer cell line is either transiently or stably transfected with a plasmid that comprises AAV rep and/or cap encoding sequences. In some aspects, the producer cell line further comprises AAV helper function encoding sequences.

In one embodiment, provided herein are methods for producing adeno-associated viral (AAV) particles comprising culturing a producer cell line as disclosed herein under condition allowing production and/or secretion of the AAV particles. In some aspects, the producer cell line produces AAV particles having packaged therein the heterologous nucleic acid sequence(s). In some aspects, the methods comprise transfecting the cells with a first plasmid encoding AAV rep and/or cap, a second plasmid encoding AAV helper function sequences, and a third plasmid encoding a transgene at a ratio of 1 to 1 to 1, 1 to 1 to 1.5, 1 to 1 to 2, or 1 to 1.5 to 1.5. The AAV helper function sequences may include a sequence encoding adenovirus E4, a sequence encoding adenovirus E2A, and/or a sequence encoding adenovirus VA RNA. In some aspects, the AAV particles are produced in greater amounts by the producer cell line than a control cell line. In some aspects, the methods further comprise isolating or purifying the AAV particles from the cell culture.

In one embodiment, provided herein are populations of adeno-associated viral (AAV) particles obtainable from a producer cell line as disclosed herein or by a method disclosed herein. In some aspects, the AAV particles are of an AAV1, AAV2, and/or AAV9 serotype. In some aspects, the AAV particles are used in the manufacture of a medicament for the treatment of a disease in a mammal.

In one embodiment, provided herein are pharmaceutical composition comprising AAV particles obtainable from a producer cell line as disclosed herein or by a method disclosed herein, and a pharmaceutically acceptable carrier. In some aspect, the pharmaceutical composition is for use in treating a disease in a mammal.

In one embodiment, provided herein are methods of treating a disease in a mammal comprising administering a pharmaceutical composition as disclosed herein to the mammal.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C: Staining with an antibody against intact AAV2 particles enables FACS-based identification of assembled AAV vectors. (FIGS. 1A and 1B) Antibody 03-61055 specifically recognizes intact AAV particles. HEK 293 cells were transfected in one of the following three conditions: a triple-transfection sufficient to produce assembled AAV vectors (orange), transfection of only the single RepCap plasmid (green), or a mock transfection with no DNA (grey). After 48 hours, these cells were fixed, stained with Antibody 03-61055 at a 1:50 dilution and the secondary antibody A-11001 at 1:200, and then subjected to FACS. The FACS plots (FIG. 1A) and histogram (FIG. 1B) show that antibody 03-61055 does not recognize cells when RepCap is expressed alone and thus the AAV capsid is not assembled. (FIG. 1C) A CRISPR/Cas9 knockout screen was performed to identify genes that regulate AAV capsid production. HEK 293 cells expressing Cas9 were transfected with the Brunello genome-wide CRISPR knockout library at a low multiplicity of infection. Following antibiotic selection and expansion, the AAV triple transfection was performed. 72 hours post-transfection, the cells were fixed and stained for assembled AAV vector. FACS was used to isolate the cells with the highest and lowest levels of AAV. These populations were then sequenced using next-generation sequencing. Subsequent data analysis identified genes that, when knocked out, affect AAV vector production.

FIGS. 2A-2B: CRISPR/Cas9 knockout screens identify potential modifiers of AAV production. Volcano plots of the results from the two CRISPR/Cas9 knockout screens, with the top hits labeled. (FIG. 2A) A volcano plot of the results from the genome-wide screen in HEK 293 cells, with the top hits labeled. On the depletion arm (in which gene knockout reduced AAV production), there are multiple members of the heparan sulfate biosynthesis pathway (EXT1, EXT2, NDST1, SLC35B3, B3GAT3, B4GALT7, B3GALT6). Another top his is KIAA0319L, which is the AAV Receptor (AAVR). On the enrichment arm (in which gene knockout increased AAV production), no single pathway emerges. (FIG. 2B) A secondary screen was performed using the top several hundred hits from the original genome-wide screen. This secondary screen was performed in a clonal AAVR knockout HEK 293 cell line in order to minimize AAV reinfection. This screen reshuffled some of the top hits and helped to reduce false positives. This secondary screen confirmed that the heparan sulfate biosynthesis pathway, shown in blue, affects AAV production.

FIGS. 3A-3D: Knockout of genes in the heparan sulfate biosynthesis pathway reduces AAV vector production. Polyclonal B3GAT3, EXT1, and B4GALT7 knockout lines were generated from wildtype HEK 293 cells. These were three of the top hits from the heparan sulfate biosynthesis pathway. As a control, wildtype HEK 293 cells expressing cas9 or polyclonal CLYBL knockout cells were used. (FIGS. 3A, 3C, 3D) Polyclonal B3GAT3 knockout cells and Polyclonal EXT1 knockout cells underwent the AAV triple transfection followed by either qPCR (n=1; FIG. 3A) or FACS (FIGS. 3C, 3D). Both B3GAT3 knockout and EXT1 knockout reduced AAV production compared to control cells. (FIG. 3B) Polyclonal B4GALT7 cell lines were generated from wildtype HEK 293. These cells underwent the AAV triple transfection followed by FACS (n=1) analysis. These preliminary results suggest that B4GALT7 knockout reduces AAV expression compared to the control CLYBL knockout cells.

FIGS. 4A-4F: Further validation supports a role for the top screen hits in AAV vector production. Polyclonal knockout lines were generated for the top three hits predicted to increase AAV production upon knockout (MON2, TMED10, TMED2). As a control, wildtype HEK 293 expressing cas9 or polyclonal CLYBL knockout cells were used. (FIG. 4A) Polyclonal TMED2 and TMED10 knockout cell lines underwent AAV triple transfection followed by qPCR analysis (n=1). Both TMED2 knockout and TMED10 knockout increased AAV production compared to the controls. (FIGS. 4B-4D) Polyclonal TMED2 (FIG. 4B), TMED10 (FIG. 4C), and MON2 (FIG. 4D) knockout cells underwent AAV triple transfection followed by FACS 48 hours post-transfection. (FIG. 4B) TMED2 knockout increased AAV production compared to wildtype cells. (FIG. 4C) For the polyclonal TMED10 knockout cells, one of the two sgRNAs tested showed an increase in AAV production compared to either wildtype cells or CLYBL knockout cells. (FIG. 4D) Polyclonal MON2 knockout cells led to an increase in in AAV production compared to control cells. (FIG. 4E) Clonal TMED10 knockout lines and clonal MON2 knockout lines were used for AAV2 production, with three replicates per line. The TMED10 and MON2 knockout clonal lines all had increased vector yields compared to the wildtype HEK 293 cells. (FIG. 4F) A clonal TMED10 knockout line showed increased AAV9 vector production compared to wildtype HEK 293 cells (n=3). This suggests that the top hits from our screen may increase AAV vector production for multiple AAV serotypes.

FIGS. 5A-5B: Ranked lists of genes that may regulate AAV vector production. Tables showing the top hits from the secondary screen in AAVR knockout cells with their corresponding fold-changes and p values. (FIG. 5A) Table showing the top 50 genes that increased AAV production when knocked out. (FIG. 5B) Table showing the top 50 genes that decreased AAV production when knocked out.

FIGS. 6A-6C: Further analysis of TMED10 knockout cell lines in AAV vector production. AAV virus was produced by triple plasmid transfection at small scale (12-well format). Following DNase treatment and lysis, qPCR was performed to quantify the vector yield. The vector yield was normalized for cell count and transfection efficiency. Fold change was calculated by setting the yield from wild-type HEK293 to 1. (FIG. 6A) AAV2 production in clonal TMED10 knockout lines as assayed by qPCR. Three to four replicates per line. (FIG. 6B) The Clone F data from FIG. 6A. Four replicates per line. (FIG. 6C) Western blot showing TMED10 protein levels in the clones.

FIGS. 7A-7B: Medium scale AAV2 production in TMED10 knockout cell line. The virus was purified by Iodixanol. (FIG. 7A) Western blot against VP proteins. (FIG. 7B) Alkaline gel electrophoresis for genome integrity.

FIGS. 8A-8B: Modification of the molar ratio for the three plasmid transfections. (FIG. 8A) Fold change in vector yield when the equimolar ratio in HEK293 cells was set to 1. (FIG. 8B). Fold change in vector yield when the respective ration in HEK293 cells was set to 1. For both figures, in each group of four bars, they represent, from left to right, AAV2_Rep_Cap: Transgene: AdHelper (1:1:1), AAV2_Rep_Cap: Transgene: AdHelper (1:2:1), AAV2_Rep_Cap: Transgene: AdHelper (1:1.5:1), and AAV2_Rep_Cap: Transgene: AdHelper (1:1.5:1.5).

### DETAILED DESCRIPTION

AAV-mediated gene therapy is a growing field with the potential to treat numerous life-threatening human diseases. A major limitation for any AAV-mediated gene therapy is the difficulty in scaling AAV vector production for commercial use. AAV vectors are typically produced by triply transfecting HEK 293 cells with two helper AAV plasmids and the gene of interest followed by harvesting AAV particles from these cells. Provided herein are genetically modified HEK 293 cells for increased AAV vector production. A genome-wide CRISPR/Cas9 knockout screen was performed to identify genes that affect the amount of AAV produced in each cell. This screen resulted in a list of the top-ranked genes that, when knocked out, modulate AAV vector production. To validate these results, a secondary screen was performed with the top several hundred gene hits to verify the reproducibility of hits from the primary genome-wide screen. Importantly, the secondary screen was performed in a clonal HEK 293 cell line where the AAV receptor (AAVR) was knocked out, to ensure that the measured increase in AAV particles in each cell is due to increased production and not reinfection from particles secreted into the media. One-by-one validation of the top gene hits was also performed to demonstrate that generation of cell lines with single gene knockouts, selected from the screen results, can provide cell lines with altered AAV production capacity. Out of the genes that were found to decrease AAV production when knocked out, the heparan sulfate pathway stood out, as it was previously associated with AAV infection. Increasing the expression of these genes is expected to have the opposite effect on AAV production. Also identified were several genes that, when knocked out, result in an increase in AAV production. Clonal cell lines where these genes are knocked out can be used to increase AAV production.

### I. ADENO-ASSOCIATED VIRAL (AAV) VECTORS

Adeno-associated virus (AAV) is a small nonpathogenic virus of the parvoviridae family. To date, numerous serologically distinct AAVs have been identified, and more than a dozen have been isolated from humans or primates. AAV is distinct from other members of this family by its dependence upon a helper virus for replication.

AAV genomes can exist in an extrachromosomal state without integrating into host cellular genomes; possess a broad host range; transduce both dividing and non-dividing cells in vitro and in vivo and maintain high levels of expression of the transduced genes. AAV viral particles are heat stable; resistant to solvents, detergents, changes in pH, and temperature; and can be column purified and/or concentrated on CsCl gradients or by other means. The AAV genome comprises a single-stranded deoxyribonucleic acid (ssDNA), either positive- or negative-sensed. The approximately 4.7 kb genome of AAV consists of one segment of single stranded DNA of either plus or minus polarity. The ends of the genome are short inverted terminal repeats (ITRs) that can fold into hairpin structures and serve as the origin of viral DNA replication.

An AAV “genome” refers to a recombinant nucleic acid molecule that is ultimately packaged or encapsulated to form an AAV particle. An AAV particle often comprises an AAV genome packaged with AAV capsid proteins. In cases where recombinant plasmids are used to construct or manufacture recombinant vectors, the AAV vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for plasmid propagation and production, but is not itself packaged or encapsulated into viral particles. Thus, an AAV vector “genome” refers to the nucleic acid molecule that is packaged or encapsulated by AAV capsid proteins.

The AAV virion (or particle) is a non-enveloped, icosahedral particle approximately 25 nm in diameter that comprises an AAV capsid. The AAV particle comprises an icosahedral symmetry comprised of three related capsid proteins, VP1, VP2 and VP3, which interact together to form the capsid. The genome of most native AAVs often contain two open reading frames (ORFs), sometimes referred to as a left ORF and a right ORF. The right ORF often encodes the capsid proteins VP1, VP2, and VP3. These proteins are often found in a ratio of 1:1:10, respectively, but may be in varied ratios, and are all derived from the right-hand ORF. The VP1, VP2, and VP3 capsid proteins differ from each other by the use of alternative splicing and an unusual start codon. Deletion analysis has shown that removal or alteration of VP1, which is translated from an alternatively spliced message, results in a reduced yield of infectious particles. Mutations within the VP3 coding region result in the failure to produce any single-stranded progeny DNA or infectious particles. The genome of an AAV particle may encode one, two, or all three of the VP1, VP2, and VP3 polypeptides.

The left ORF often encodes the non-structural Rep proteins, Rep 40, Rep 52, Rep 68, and Rep 78, which are involved in regulation of replication and transcription in addition to the production of single-stranded progeny genomes. Two of the Rep proteins have been associated with the preferential integration of AAV genomes into a region of the q arm of human chromosome 19. Rep68/78 have been shown to possess NTP binding activity as well as DNA and RNA helicase activities. Some Rep proteins possess a nuclear localization signal as well as several potential phosphorylation sites. The genome of an AAV (e.g., an rAAV) may encode some or all of the Rep proteins. The genome of an AAV (e.g., an rAAV) may not encode the Rep proteins. One or more of the Rep proteins can be delivered in trans and are therefore not included in an AAV particle comprising a nucleic acid encoding a recombinant polypeptide.

The ends of the AAV genome comprise short inverted terminal repeats (ITR) that can fold into T-shaped hairpin structures to serve as the origin of viral DNA replication. Accordingly, the genome of an AAV comprises one or more (e.g., a pair of) ITR sequences that flank a single stranded viral genomic DNA molecule. The ITR sequences often have a length of about 145 bases each. The ITR region has a GAGC repeat motif and the terminal resolution site (trs). The repeat motif can bind Rep when the ITR is in either a linear or hairpin conformation. This binding is thought to position Rep68/78 for cleavage at the trs which occurs in a site- and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

The term “recombinant,” as a modifier of vector, such as a recombinant AAV vector, as well as a modifier of sequences such as recombinant nucleic acid sequences and polypeptides, means that the compositions have been manipulated (i.e., engineered) in a fashion that generally does not occur in nature. A particular example of a recombinant vector, such as an AAV vector, would be where a nucleic acid sequence that is not normally present in the wild-type viral genome is inserted within the viral genome. An example of a recombinant nucleic acid sequence would be where a nucleic acid (e.g., gene) encodes an inhibitory RNA cloned into a vector, with or without 5′, 3′ and/or intron regions that the gene is normally associated within the viral genome. Although the term “recombinant” is not always used herein in reference to vectors, such as viral vectors, as well as sequences such as polynucleotides, “recombinant” forms including nucleic acid sequences, polynucleotides, transgenes, etc. are expressly included in spite of any such omission.

A recombinant viral “vector” is derived from the wild type genome of a virus by using molecular methods to remove part of the wild type genome from the virus, and replacing it with a non-native nucleic acid sequence. Typically, for example, for AAV, one or both inverted terminal repeat (ITR) sequences of the AAV genome are retained in the recombinant AAV vector. A “recombinant” viral vector (e.g., rAAV vector) is distinguished from a viral (e.g., AAV) genome, since part of the viral genome has been replaced with a non-native sequence with respect to the viral genomic nucleic acid such a nucleic acid encoding a transactivator or nucleic acid encoding an inhibitory RNA or nucleic acid encoding a therapeutic protein. Incorporation of such non-native nucleic acid sequences therefore defines the viral vector as a “recombinant” vector, which in the case of AAV can be referred to as an “rAAV vector.”

In certain embodiments, an AAV (e.g., an rAAV) comprises two ITRs. In certain embodiments, an AAV (e.g., an rAAV) comprises a pair of ITRs. In certain embodiments, an AAV (e.g., an rAAV) comprises a pair of ITRs that flank (i.e., are at each 5′ and 3′ end) of a nucleic acid sequence that at least encodes a polypeptide having function or activity.

An AAV vector (e.g., rAAV vector) can be packaged and is referred to herein as an “AAV particle” for subsequent infection (transduction) of a cell, ex vivo, in vitro or in vivo. Where a recombinant AAV vector is encapsulated or packaged into an AAV particle, the particle can also be referred to as an “rAAV particle.” In certain embodiments, an AAV particle is an rAAV particle. An rAAV particle often comprises an rAAV vector, or a portion thereof. An rAAV particle can be one or more rAAV particles (e.g., a plurality of AAV particles). rAAV particles typically comprise proteins that encapsulate or package the rAAV vector genome (e.g., capsid proteins). It is noted that reference to an rAAV vector can also be used to reference an rAAV particle.

Any suitable AAV particle (e.g., rAAV particle) can be used for a method or use herein. An rAAV particle, and/or genome comprised therein, can be derived from any suitable serotype or strain of AAV. An rAAV particle, and/or genome comprised therein, can be derived from two or more serotypes or strains of AAV. Accordingly, an rAAV can comprise proteins and/or nucleic acids, or portions thereof, of any serotype or strain of AAV, wherein the AAV particle is suitable for infection and/or transduction of a mammalian cell. Non-limiting examples of AAV serotypes include AAV1, AAV2, AAV3, AAV4, AAV5, AAV6, AAV7, AAV8, AAV9, AAV10, AAV11, AAV12, AAV-rh74, AAV-rh10, and AAV-2i8.

In certain embodiments, a plurality of rAAV particles comprises particles of, or derived from, the same strain or serotype (or subgroup or variant). In certain embodiments a plurality of rAAV particles comprise a mixture of two or more different rAAV particles (e.g., of different serotypes and/or strains).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

In certain embodiments, an rAAV vector based upon a first serotype genome corresponds to the serotype of one or more of the capsid proteins that package the vector. For example, the serotype of one or more AAV nucleic acids (e.g., ITRs) that comprises the AAV vector genome corresponds to the serotype of a capsid that comprises the rAAV particle.

In certain embodiments, an rAAV vector genome can be based upon an AAV (e.g., AAV2) serotype genome distinct from the serotype of one or more of the AAV capsid proteins that package the vector. For example, an rAAV vector genome can comprise AAV2 derived nucleic acids (e.g., ITRs), whereas at least one or more of the three capsid proteins are derived from a different serotype, e.g., an AAV1, AAV3, AAV4, AAV5, AAV6, AAV7, AAV8, AAV9, AAV10, AAV11, AAV12, Rh10, Rh74, or AAV-2i8 serotype or variant thereof.

In certain embodiments, an rAAV particle or a vector genome thereof related to a reference serotype has a polynucleotide, polypeptide or subsequence thereof that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a polynucleotide, polypeptide or subsequence of an AAV1, AAV2, AAV3, AAV4, AAV5, AAV6, AAV7, AAV8, AAV9, AAV10, AAV11, AAV12, Rh10, Rh74, or AAV-2i8 particle. In particular embodiments, an rAAV particle or a vector genome thereof related to a reference serotype has a capsid or ITR sequence that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a capsid or ITR sequence of an AAV1, AAV2, AAV3, AAV4, AAV5, AAV6, AAV7, AAV8, AAV9, AAV10, AAV11, AAV12, Rh10, Rh74, or AAV-2i8 serotype.

In certain embodiments, a method herein comprises manufacture, use, administration, or delivery of an rAAV1, rAAV2, rAAV3, rAAV4, rAAV5, rAAV6, rAAV7, rAAV8, rAAV9, rAAV10, rAAV11, rAAV12, rRh10, rRh74, or rAAV-2i8 particle.

In certain embodiments an rAAV2 particle comprises an AAV2 capsid. In certain embodiments an rAAV2 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments an rAAV2 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments, an rAAV2 particle is a variant of a native or wild-type AAV2 particle. In some aspects, one or more capsid proteins of an AAV2 variant have 1, 2, 3, 4, 5, 5-10, 10-15, 15-20 or more amino acid substitutions compared to capsid protein(s) of a native or wild-type AAV2 particle.

In certain embodiments, an rAAV9 particle comprises an AAV9 capsid. In certain embodiments an rAAV9 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments an rAAV9 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments, an rAAV9 particle is a variant of a native or wild-type AAV9 particle. In some aspects, one or more capsid proteins of an AAV9 variant have 1, 2, 3, 4, 5, 5-10, 10-15, 15-20 or more amino acid substitutions compared to capsid protein(s) of a native or wild-type AAV9 particle.

In certain embodiments, an rAAV particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV1, AAV2, AAV3, AAV4, AAV5, AAV6, AAV7, AAV8, AAV9, AAV10, AAV11, AAV12, AAV-rh74, AAV-rh10, or AAV-2i8, as long as they retain one or more desired ITR functions (e.g., ability to form a hairpin, which allows DNA replication; integration of the AAV DNA into a host cell genome; and/or packaging, if desired).

An rAAV particle can comprise an ITR having any suitable number of “GAGC” repeats. In certain embodiments, an ITR of an AAV2 particle comprises 1, 2, 3, 4, 5, 6, 7, 8, 9 or 10 or more “GAGC” repeats. In certain embodiments, an rAAV2 particle comprises an ITR comprising three “GAGC” repeats. In certain embodiments, an rAAV2 particle comprises an ITR which has less than four “GAGC” repeats. In certain embodiments, an rAAV2 particle comprises an ITR which has more than four “GAGC” repeats. In certain embodiments an ITR of an rAAV2 particle comprises a Rep binding site wherein the fourth nucleotide in the first two “GAGC” repeats is a C rather than a T.

Exemplary suitable length of DNA can be incorporated in rAAV vectors for packaging/encapsidation into a rAAV particle can about 5 kilobases (kb) or less. In particular, embodiments, length of DNA is less than about 5 kb, less than about 4.5 kb, less than about 4 kb, less than about 3.5 kb, less than about 3 kb, or less than about 2.5 kb.

rAAV vectors that include a nucleic acid sequence that directs the expression of an RNAi or polypeptide can be generated using suitable recombinant techniques known in the art (e.g., see Sambrook et al., 1989). Recombinant AAV vectors are typically packaged into transduction-competent AAV particles and propagated using an AAV viral packaging system. A transduction-competent AAV particle is capable of binding to and entering a mammalian cell and subsequently delivering a nucleic acid cargo (e.g., a heterologous gene) to the nucleus of the cell. Thus, an intact rAAV particle that is transduction-competent is configured to transduce a mammalian cell. An rAAV particle configured to transduce a mammalian cell is often not replication competent, and requires additional protein machinery to self-replicate. Thus, an rAAV particle that is configured to transduce a mammalian cell is engineered to bind and enter a mammalian cell and deliver a nucleic acid to the cell, wherein the nucleic acid for delivery is often positioned between a pair of AAV ITRs in the rAAV genome.

Suitable host cells for producing transduction-competent AAV particles include but are not limited to microorganisms, yeast cells, insect cells, and mammalian cells that can be, or have been, used as recipients of a heterologous rAAV vectors. Cells from the stable human cell line, HEK293 (readily available through, e.g., the American Type Culture Collection under Accession Number ATCC CRL1573) can be used. In certain embodiments a modified human embryonic kidney cell line (e.g., HEK293), which is transformed with adenovirus type-5 DNA fragments, and expresses the adenoviral Ela and Elb genes is used to generate recombinant AAV particles. The modified HEK293 cell line is readily transfected, and provides a particularly convenient platform in which to produce rAAV particles.

Provided herein are genetically modified HEK 293 cells for increased AAV vector production. These cells may be modified to knock out a gene in order to provide for increased AAV production. For example, one or more of the following genes may be knocked out: MON2, TMED10, TMED2, HS2ST1, C3orf58, SPPL3, SURF4, LSM5, ARF1, and PI4 KB. Clonal cell lines where one or more of these genes are knocked out can be used to increase AAV production. On the other hand, these cells may be modified to increase the expression of a gene in order to provide for increase AAV production. For example, expression of one or more of the following genes may be increased: B4GALT7, B3GAT3, OAF, EXT2, COMMD3, SLC35D1, B3GALT6, SDC1, NDST1, RAC1, CSK, GLCE, PDCL, FAM20B, TM4SF5, DGAT2, POMT1, YY1, and DPF2.

In certain embodiments, AAV helper functions are introduced into the host cell by transfecting the host cell with an AAV helper construct either prior to, or concurrently with, the transfection of an AAV vector. AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products. In a preferred embodiment, a triple transfection method (described in detail in U.S. Pat. Nos. 6,001,650 and 7,261,544, each incorporated by reference herein in its entirety) is used to produce rAAV virions.

### II. MODULATION OF GENE EXPRESSION

Provided herein are systems for increasing AAV particle production. These systems comprise producer cell lines adapted for the production of AAV vectors, as well as methods of producing AAV vectors using said producer cell lines. Also provided are AAV particles produced by said production systems, producer cell lines and methods.

Accordingly, provide herein are methods of producing an AAV particle comprising modulating the expression of one or more genes and/or proteins in a producer cell line. Some aspects of the present disclosure relate to modulating the expression level of various genes in genetically modified HEK 293 cells. For example, the producer cell may be genetically engineered to reduce or eliminate the expression of MON2 (NCBI Entrez Gene: 23041), TMED10 (NCBI Entrez Gene: 10972), TMED2 (NCBI Entrez Gene: 10959), HS2ST1 (NCBI Entrez Gene: 9653), C3orf58, SPPL3 (NCBI Entrez Gene: 121665), SURF4 (NCBI Entrez Gene: 6836), LSM5 (NCBI Entrez Gene: 23658), ARF1 (NCBI Entrez Gene: 375), and/or PI4 KB (NCBI Entrez Gene: 5298), and/or increase the expression of B4GALT7 (NCBI Entrez Gene: 11285), B3GAT3 (NCBI Entrez Gene: 26229), OAF (NCBI Entrez Gene: 220323), EXT2 (NCBI Entrez Gene: 2132), COMMD3 (NCBI Entrez Gene: 23412), SLC35D1 (NCBI Entrez Gene: 23169), B3GALT6 (NCBI Entrez Gene: 126792), SDC1 (NCBI Entrez Gene: 6382), NDST1 (NCBI Entrez Gene: 3340), RAC1 (NCBI Entrez Gene: 5879), CSK (NCBI Entrez Gene: 1445), GLCE (NCBI Entrez Gene: 26035), PDCL (NCBI Entrez Gene: 5082), FAM20B (NCBI Entrez Gene: 9917), TM4SF5 (NCBI Entrez Gene: 9032), DGAT2 (NCBI Entrez Gene: 84649), POMT1 (NCBI Entrez Gene: 10585), YY1 (NCBI Entrez Gene: 7528), and/or DPF2 (NCBI Entrez Gene: 5977). All of the foregoing NCBI Entrez Gene entries are incorporated by reference herein in their entirety. Such modulation may be effected, for example, through the use of inhibitory RNAs or CRISPR systems, or any other known method of genetic engineering.

Modulation may be increasing or reducing (decreasing) the expression of the one or more gene and/or protein. In cases where multiple genes and/or proteins are modulated, all the gene/proteins may be increased, or all the genes/proteins may be decrease, or one or more genes/proteins may be increased and others of the genes/proteins may be decreased. In a preferred embodiment, the modulation is decreasing the expression of the one or more gene and/or protein.

Modulation, whether an increase or a reduction in the expression of one or more gene and/or protein in a producer cell line, may be measured relative to a control. Thus, the expression of one or more gene and/or protein in a genetically modified producer cell line may be compared with the expression of said one or more gene and/or protein in a control. The actual amount of the one or more gene and/or protein, such as the mass, molar amount, concentration or molarity of the one or more gene and/or protein in the genetically modified producer cell line and the control may be assessed and compared with the corresponding value from the control. Alternatively, the expression of one or more gene and/or protein in a genetically modified producer cell line may be compared with that of the control without quantifying the mass, molar amount, concentration or molarity of the one or more gene and/or protein.

The control may be an equivalent producer cell line in which no modulation of the one or more gene and/or protein has been effected. For example, in the case where the genetically modified producer cell line is a transgenic cell line in which MON2 expression has been reduced or eliminated, a suitable control would be the same cell line in which MON2 expression has not been altered. Such control cell lines may be wild-type cell lines. A control method typically uses a control producer cell line as described herein. Conventional methods for the production of AVV, including known methods may be considered control methods.

The expression of the one or more gene and/or protein in a genetically modified producer cell line may differ by at least 10%, at least 20%, at least 30%, at least 40%, at least 50%, at least 60, at least 70%, at least 80%, at least 90%, at least 100%, at least 150%, at least 200% or more compared with a control.

For example, if the expression of one or more gene and/or protein in a genetically modified producer cell line is reduced compared with a control, the expression may be reduced partially or totally compared with the control. Typically the expression is reduced by at least 10%, at least 20%, at least 30%, at least 40%, at least 50%, at least 60, at least 70%, at least 80%, at least 90%, at least 95%, at least 99%, up to total elimination (knockout) of expression of the one or more gene and/or protein.

If the expression of one or more gene and/or protein in a genetically modified producer cell line is increased compared with a control, the expression may be increased by at least 10%, at least 20%, at least 30%, at least 40%, at least 50%, at least 60, at least 70%, at least 80%, at least 90&, at least 100%, at least 150%, at least 200% compared with the control.

The expression of one or more gene and/or protein in a genetically modified producer cell line may be determined by quantitative and/or qualitative analysis. Gene expression may be expressed in terms of mRNA levels or protein levels.

The expression level of the one/or more gene and/or protein in a genetically modified producer cell line encompasses the mass of the one/or more gene and/or protein, the molar amount of the one/or more gene and/or protein, the concentration of the one/or more gene and/or protein and the molarity of the one/or more gene and/or protein. This expression level may be given in any appropriate units. For example, the concentration of the one or more gene and/or protein may be given in pg/ml, ng/ml or μg/ml.

The expression level of the one/or more gene and/or protein in a genetically modified producer cell line may be measured directly or indirectly. The relative expression of the one or more modulated gene and/or protein in a genetically modified producer cell line relative to a control may be determined using any appropriate technique. Suitable standard techniques are known in the art, for example Western blotting and enzyme-linked immunosorbent assays (ELISAs).

The expression level of the one or more gene and/or protein to be modulated may be altered compared with a control for at least 12 hours, at least 24 hours, at least 30 hours, at least 48 hours, at least 72 hours, at least 96 hours, at least 120 hours, at least 144 hours, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks or more.

The expression level of the one or more gene and/or protein to be modulated may be altered compared with a control for at least one, at least two, at least three, at least four, at least five, at least ten, at least 20, at least 30, at least 40 or more passages of the producer cell line in culture. The expression level of the one or more gene and/or protein to be modulated may be altered indefinitely.

### A. Inhibitory RNAs

“RNA interference (RNAi)” is the process of sequence-specific, post-transcriptional gene silencing initiated by siRNA. During RNAi, siRNA induces degradation of target mRNA with consequent sequence-specific inhibition of gene expression.

An “inhibitory RNA,” “RNAi,” “small interfering RNA” or “short interfering RNA” or “siRNA” molecule, “short hairpin RNA” or “shRNA” molecule, or “miRNA” is an RNA duplex of nucleotides that is targeted to a nucleic acid sequence of interest. As used herein, the term “siRNA” is a generic term that encompasses the subset of shRNAs and miRNAs. An “RNA duplex” refers to the structure formed by the complementary pairing between two regions of an RNA molecule. siRNA is “targeted” to a gene in that the nucleotide sequence of the duplex portion of the siRNA is complementary to a nucleotide sequence of the targeted gene. In certain embodiments, the siRNAs are targeted to the sequence encoding huntingtin. In some embodiments, the length of the duplex of siRNAs is less than 30 base pairs. In some embodiments, the duplex can be 29, 28, 27, 26, 25, 24, 23, 22, 21, 20, 19, 18, 17, 16, 15, 14, 13, 12, 11 or 10 base pairs in length. In some embodiments, the length of the duplex is 19 to 25 base pairs in length. In certain embodiment, the length of the duplex is 19 or 21 base pairs in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24 or 25 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

shRNAs are comprised of stem-loop structures which are designed to contain a 5′ flanking region, siRNA region segments, a loop region, a 3′ siRNA region and a 3′ flanking region. Most RNAi expression strategies have utilized short-hairpin RNAs (shRNAs) driven by strong polIII-based promoters. Many shRNAs have demonstrated effective knock down of the target sequences in vitro as well as in vivo, however, some shRNAs which demonstrated effective knock down of the target gene were also found to have toxicity in vivo.

miRNAs are small cellular RNAs (˜22 nt) that are processed from precursor stem loop transcripts. Known miRNA stem loops can be modified to contain RNAi sequences specific for genes of interest. miRNA molecules can be preferable over shRNA molecules because miRNAs are endogenously expressed. Therefore, miRNA molecules are unlikely to induce dsRNA-responsive interferon pathways, they are processed more efficiently than shRNAs, and they have been shown to silence 80% more effectively.

A recently discovered alternative approach is the use of artificial miRNAs (pri-miRNA scaffolds shuttling siRNA sequences) as RNAi vectors. Artificial miRNAs more naturally resemble endogenous RNAi substrates and are more amenable to Pol-II transcription (e.g., allowing tissue-specific expression of RNAi) and polycistronic strategies (e.g., allowing delivery of multiple siRNA sequences). See U.S. Pat. No. 10,093,927, which is incorporated by reference.

The transcriptional unit of a “shRNA” is comprised of sense and antisense sequences connected by a loop of unpaired nucleotides. shRNAs are exported from the nucleus by Exportin-5, and once in the cytoplasm, are processed by Dicer to generate functional siRNAs. “miRNAs” stem-loops are comprised of sense and antisense sequences connected by a loop of unpaired nucleotides typically expressed as part of larger primary transcripts (pri-miRNAs), which are excised by the Drosha-DGCR8 complex generating intermediates known as pre-miRNAs, which are subsequently exported from the nucleus by Exportin-5, and once in the cytoplasm, are processed by Dicer to generate functional siRNAs. “Artificial miRNA” or an “artificial miRNA shuttle vector”, as used herein interchangeably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (˜35 nucleotides upstream and ˜40 nucleotides downstream) arise from restriction enzyme sites within the multiple cloning site of the siRNA. As used herein the term “miRNA” encompasses both the naturally occurring miRNA sequences as well as artificially generated miRNA shuttle vectors.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal or a sequence of six Ts.

In designing RNAi there are several factors that need to be considered, such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Preferably the siRNA exhibits greater than 80%, 85%, 90%, 95%, 98%, or even 100% identity between the sequence of the siRNA and the gene to be inhibited. Sequences less than about 80% identical to the target gene are substantially less effective. Thus, the greater homology between the siRNA and the gene to be inhibited, the less likely expression of unrelated genes will be affected.

In addition, the size of the siRNA is an important consideration. In some embodiments, siRNA molecules may include at least about 19-25 nucleotides and are able to modulate gene expression. An siRNA may be preferably less than 500, 200, 100, 50, or 25 nucleotides in length. More preferably, the siRNA is from about 19 nucleotides to about 25 nucleotides in length.

A siRNA target generally means a polynucleotide comprising a region that encodes a polypeptide, or a polynucleotide region that regulates replication, transcription, or translation or other processes important to expression of the polypeptide, or a polynucleotide comprising both a region that encodes a polypeptide and a region operably linked thereto that regulates expression. Any gene being expressed in a cell can be targeted.

### B. CRISPR Systems

Gene editing is a technology that allows for the modification of target genes within living cells. Recently, harnessing the bacterial immune system of CRISPR to perform on demand gene editing revolutionized the way scientists approach genomic editing. The Cas9 protein of the CRISPR system, which is an RNA guided DNA endonuclease, can be engineered to target new sites with relative ease by altering its guide RNA sequence. This discovery has made sequence specific gene editing functionally effective.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor (e.g., KRAB) or activator, to affect gene expression. Alternatively, a CRISPR system with a catalytically inactivate Cas9 further comprises a transcriptional repressor or activator fused to a ribosomal binding protein.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence.” In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. The Cas enzyme may be a target gene under the control of a regulated alternative splicing event, as disclosed herein, either as a chimeric target gene minigene or as a target gene for a chimeric minigene transactivator. The gRNA may be under the control of a constitutive promoter.

Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx 10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules.

The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US20110059502, incorporated herein by reference.

### III. PHARMACEUTICAL COMPOSITIONS

In certain embodiments, a method herein comprises use, administration, or delivery of an AAV particle produced using the genetically engineered producer cells lines described herein. As such, the AAV particles may be formulated into a pharmaceutical composition.

As used herein the term “pharmaceutically acceptable” and “physiologically acceptable” mean a biologically acceptable composition, formulation, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Such composition, “pharmaceutically acceptable” and “physiologically acceptable” formulations and compositions can be sterile. Such pharmaceutical formulations and compositions may be used, for example in administering a viral particle to a subject.

Such formulations and compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Supplementary active compounds (e.g., preservatives, antibacterial, antiviral and antifungal agents) can also be incorporated into the formulations and compositions.

Pharmaceutical compositions typically contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as surfactants, wetting or emulsifying agents, pH buffering substances, and the like, may be present in such vehicles.

Pharmaceutical compositions can be formulated to be compatible with a particular route of administration or delivery, as set forth herein or known to one of skill in the art. Thus, pharmaceutical compositions include carriers, diluents, or excipients suitable for administration or delivery by various routes.

Pharmaceutical forms suitable for injection or infusion of viral particles can include sterile aqueous solutions or dispersions which are adapted for the extemporaneous preparation of sterile injectable or infusible solutions or dispersions, optionally encapsulated in liposomes. In all cases, the ultimate form should be a sterile fluid and stable under the conditions of manufacture, use and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Isotonic agents, for example, sugars, buffers or salts (e.g., sodium chloride) can be included. Prolonged absorption of injectable compositions can be brought about by the use in the compositions of agents delaying absorption, for example, aluminum monostearate and gelatin.

Solutions or suspensions of viral particles can optionally include one or more of the following components: a sterile diluent such as water for injection, saline solution, such as phosphate buffered saline (PBS), artificial CSF, a surfactants, fixed oils, a polyol (for example, glycerol, propylene glycol, and liquid polyethylene glycol, and the like), glycerin, or other synthetic solvents; antibacterial and antifungal agents such as parabens, chlorobutanol, phenol, ascorbic acid, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Pharmaceutical formulations, compositions and delivery systems appropriate for the compositions, methods and uses described herein are known in the art (see, e.g., Remington: The Science and Practice of Pharmacy (2003) 20^{th} ed., Mack Publishing Co., Easton, PA; Remington's Pharmaceutical Sciences (1990) 18^{th} ed., Mack Publishing Co., Easton, PA; The Merck Index (1996) 12^{th} ed., Merck Publishing Group, Whitehouse, NJ; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, MD; and Poznansky et al., Drug Delivery Systems (1980), R. L. Juliano, ed., Oxford, N.Y., pp. 253-315).

Viral particles and their compositions may be formulated in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms are dependent upon the number of viral particles believed necessary to produce the desired effect(s). The amount necessary can be formulated in a single dose, or can be formulated in multiple dosage units. The dose may be adjusted to a suitable viral particle concentration, optionally combined with an anti-inflammatory agent, and packaged for use.

In one embodiment, pharmaceutical compositions will include sufficient genetic material to provide a therapeutically effective amount, i.e., an amount sufficient to reduce or ameliorate symptoms or an adverse effect of a disease state in question or an amount sufficient to confer the desired benefit.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Thus, for example, viral particles, and pharmaceutical compositions thereof, can be packaged in single or multiple unit dosage form for ease of administration and uniformity of dosage.

Formulations containing viral particles typically contain an effective amount, the effective amount being readily determined by one skilled in the art. The viral particles may typically range from about 1% to about 95% (w/w) of the composition, or even higher if suitable. The quantity to be administered depends upon factors such as the age, weight and physical condition of the mammal or the human subject considered for treatment.

Effective dosages can be established by one of ordinary skill in the art through routine trials establishing dose response curves.

### IV. DEFINITIONS

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

An “expression vector” is a specialized vector that contains a gene or nucleic acid sequence with the necessary regulatory regions needed for expression in a host cell. An expression vector may contain at least an origin of replication for propagation in a cell and optionally additional elements, such as a heterologous nucleic acid sequence, expression control element (e.g., a promoter, enhancer), intron, ITR(s), and polyadenylation signal.

The terms “polynucleotide,” “nucleic acid” and “transgene” are used interchangeably herein to refer to all forms of nucleic acid, oligonucleotides, including deoxyribonucleic acid (DNA) and ribonucleic acid (RNA) and polymers thereof. Polynucleotides include genomic DNA, cDNA and antisense DNA, and spliced or unspliced mRNA, rRNA, tRNA and inhibitory DNA or RNA (RNAi, e.g., small or short hairpin (sh)RNA, microRNA (miRNA), small or short interfering (si) RNA, trans-splicing RNA, or antisense RNA). Polynucleotides can include naturally occurring, synthetic, and intentionally modified or altered polynucleotides (e.g., variant nucleic acid). Polynucleotides can be single stranded, double stranded, or triplex, linear or circular, and can be of any suitable length. In discussing polynucleotides, a sequence or structure of a particular polynucleotide may be described herein according to the convention of providing the sequence in the 5′ to 3′ direction.

A nucleic acid encoding a polypeptide often comprises an open reading frame that encodes the polypeptide. Unless otherwise indicated, a particular nucleic acid sequence also includes degenerate codon substitutions.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism.

A “promoter” refers to a nucleotide sequence, usually upstream (5′) of a coding sequence, which directs and/or controls the expression of the coding sequence by providing the recognition for RNA polymerase and other factors required for proper transcription. A pol II promoter includes a minimal promoter that is a short DNA sequence comprised of a TATA-box and optionally other sequences that serve to specify the site of transcription initiation, to which regulatory elements are added for control of expression. A type 1 pol III promoter includes three cis-acting sequence elements downstream of the transcriptional start site: a) 5′sequence element (A block); b) an intermediate sequence element (I block); c) 3′ sequence element (C block). A type 2 pol III promoter includes two essential cis-acting sequence elements downstream of the transcription start site: a) an A box (5′ sequence element); and b) a B box (3′ sequence element). A type 3 pol III promoter includes several cis-acting promoter elements upstream of the transcription start site, such as a traditional TATA box, proximal sequence element (PSE), and a distal sequence element (DSE).

An “enhancer” is a DNA sequence that can stimulate transcription activity and may be an innate element of the promoter or a heterologous element that enhances the level or tissue specificity of expression. It is capable of operating in either orientation (5′->3′ or 3′->5′), and may be capable of functioning even when positioned either upstream or downstream of the promoter.

Promoters and/or enhancers may be derived in their entirety from a native gene, or be composed of different elements derived from different elements found in nature, or even be comprised of synthetic DNA segments. A promoter or enhancer may comprise DNA sequences that are involved in the binding of protein factors that modulate/control effectiveness of transcription initiation in response to stimuli, physiological or developmental conditions.

Non-limiting examples of promoters include SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, actin promoter, U6, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. In addition, sequences derived from intronic miRNA promoters, such as, for example, the miR107, miR206, miR208b, miR548f-2, miR569, miR590, miR566, and miR128 promoter, will also find use herein (see, e.g., Monteys et al., 2010). Accordingly, any of the above-referenced constitutive promoters can be used to control transcription of a heterologous gene insert.

A nucleic acid/transgene is “operably linked” when it is placed into a functional relationship with another nucleic acid sequence. A nucleic acid/transgene encoding and RNAi or a polypeptide, or a nucleic acid directing expression of a polypeptide may include an inducible promoter, or a tissue-specific promoter for controlling transcription of the encoded polypeptide. A nucleic acid operably linked to an expression control element can also be referred to as an expression cassette.

A “transgene” is used herein to conveniently refer to a nucleic acid sequence/polynucleotide that is intended or has been introduced into a cell or organism. Transgenes include any nucleic acid, such as a gene that encodes an inhibitory RNA or polypeptide or protein, and are generally heterologous with respect to naturally occurring AAV genomic sequences.

Transgenes under control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting a suitable promoter (constitutive versus inducible; strong versus weak), it is possible to control both the existence and level of expression of a polypeptide in the genetically modified cell. If the gene encoding the polypeptide is under the control of an inducible promoter, delivery of the polypeptide in situ is triggered by exposing the genetically modified cell in situ to conditions for permitting transcription of the polypeptide, e.g., by intraperitoneal injection of specific inducers of the inducible promoters which control transcription of the agent. For example, in situ expression by genetically modified cells of a polypeptide encoded by a gene under the control of the metallothionein promoter, is enhanced by contacting the genetically modified cells with a solution containing the appropriate (i.e., inducing) metal ions in situ.

The term “transduce” refers to introduction of a nucleic acid sequence into a cell or host organism by way of a vector (e.g., a viral particle). Introduction of a transgene into a cell by a viral particle is can therefore be referred to as “transduction” of the cell. The transgene may or may not be integrated into genomic nucleic acid of a transduced cell. If an introduced transgene becomes integrated into the nucleic acid (genomic DNA) of the recipient cell or organism it can be stably maintained in that cell or organism and further passed on to or inherited by progeny cells or organisms of the recipient cell or organism. Finally, the introduced transgene may exist in the recipient cell or host organism extra chromosomally, or only transiently. A “transduced cell” is therefore a cell into which the transgene has been introduced by way of transduction. Thus, a “transduced” cell is a cell into which, or a progeny thereof in which a transgene has been introduced. A transduced cell can be propagated, transgene transcribed and the encoded inhibitory RNA or protein expressed. For gene therapy uses and methods, a transduced cell can be in a mammal.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation, e.g., a missense or nonsense mutation.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a protein encoded thereby, and some codons of the nucleic acid variant will be changed which in turn changes the amino acids of a protein encoded thereby.

The terms “protein” and “polypeptide” are used interchangeably herein. The “polypeptides” encoded by a “nucleic acid” or “polynucleotide” or “transgene” disclosed herein include partial or full-length native sequences, as with naturally occurring wild-type and functional polymorphic proteins, functional subsequences (fragments) thereof, and sequence variants thereof, so long as the polypeptide retains some degree of function or activity. Accordingly, in methods and uses described herein, such polypeptides encoded by nucleic acid sequences are not required to be identical to the endogenous protein that is defective, or whose activity, function, or expression is insufficient, deficient or absent in a treated mammal.

Non-limiting examples of modifications include one or more nucleotide or amino acid substitutions (e.g., about 1 to about 3, about 3 to about 5, about 5 to about 10, about 10 to about 15, about 15 to about 20, about 20 to about 25, about 25 to about 30, about 30 to about 40, about 40 to about 50, about 50 to about 100, about 100 to about 150, about 150 to about 200, about 200 to about 250, about 250 to about 500, about 500 to about 750, about 750 to about 1000 or more nucleotides or residues).

An example of an amino acid modification is a conservative amino acid substitution or a deletion. In particular embodiments, a modified or variant sequence retains at least part of a function or activity of the unmodified sequence (e.g., wild-type sequence).

Another example of an amino acid modification is a targeting peptide introduced into a capsid protein of a viral particle. Peptides have been identified that target recombinant viral vectors, to the central nervous system, such as to distinct brain regions.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein. In certain embodiments, the fragment or portion is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

“Conservative variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are identical is that one polypeptide is immunologically reactive with antibodies raised against the second polypeptide. Thus, a polypeptide is identical to a second polypeptide, for example, where the two peptides differ only by a conservative substitution.

The terms “treat” and “treatment” refer to both therapeutic treatment and prophylactic or preventative measures, wherein the object is to prevent, inhibit, reduce, or decrease an undesired physiological change or disorder, such as the development, progression or worsening of the disorder. For purposes of this invention, beneficial or desired clinical results include, but are not limited to, alleviation of symptoms, diminishment of extent of disease, stabilizing a (i.e., not worsening or progressing) symptom or adverse effect of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those predisposed (e.g., as determined by a genetic assay).

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1-Identifying Regulators of AAV Vector Production Using CRISPR/Cas9 Knockout Screens for the Generation of Cell Lines with Higher AAV Production Capacity

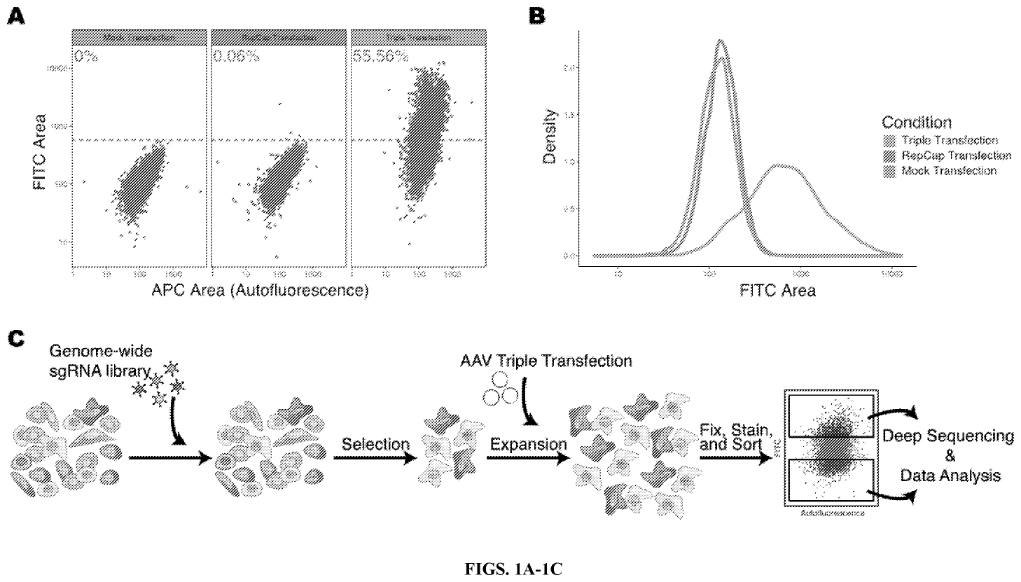

A genome-wide CRISPR/Cas9 knockout screen was performed to identify genes that affect the amount of AAV produced in each cell (FIG. 1C). HEK 293 cells expressing Cas9 were transfected with the Brunello genome-wide CRISPR knockout library at a low multiplicity of infection. Following antibiotic selection and expansion, the AAV triple transfection was performed. 72 hours post-transfection, the cells were fixed and stained for assembled AAV vector using antibody 03-61055, which specifically recognizes intact AAV particles (see FIGS. 1A-1B). FACS was used to isolate the cells with the highest and lowest levels of AAV. These populations were then sequenced using next-generation sequencing.

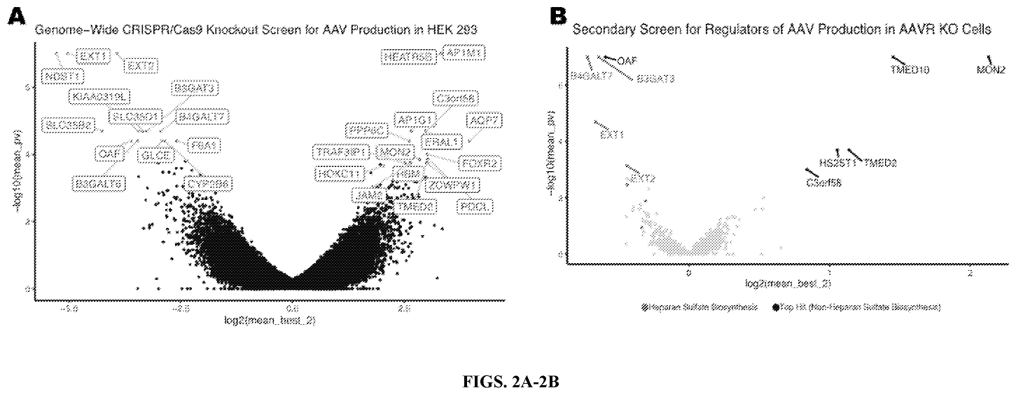

Subsequent data analysis identified genes that, when knocked out, modulate AAV vector production (FIG. 2A). On the depletion arm (in which gene knockout reduced AAV production), there were multiple members of the heparan sulfate biosynthesis pathway (EXT1, EXT2, NDST1, SLC35B3, B3GAT3, B4GALT7, B3GALT6). Another top his is KIAA0319L, which is the AAV Receptor (AAVR). On the enrichment arm (in which gene knockout increased AAV production), no single pathway emerged.

To validate these results, a secondary screen was performed with the top several hundred gene hits to verify the reproducibility of hits from the primary genome-wide screen (FIG. 2B). Importantly, the secondary screen was performed in a clonal HEK 293 cell line where the AAV receptor (AAVR) was knocked out, to ensure that the measured increase in AAV particles in each cell is due to increased production and not reinfection from particles secreted into the media. This screen reshuffled some of the top hits and helped to reduce false positives. This secondary screen confirmed that the heparan sulfate biosynthesis pathway, shown in blue in FIG. 2B, affects AAV production.

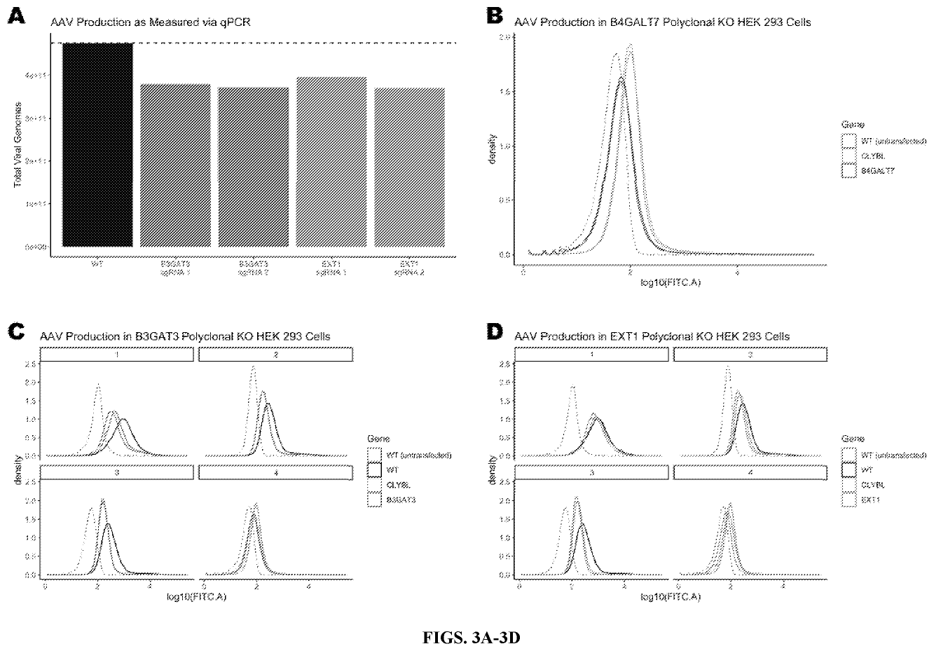

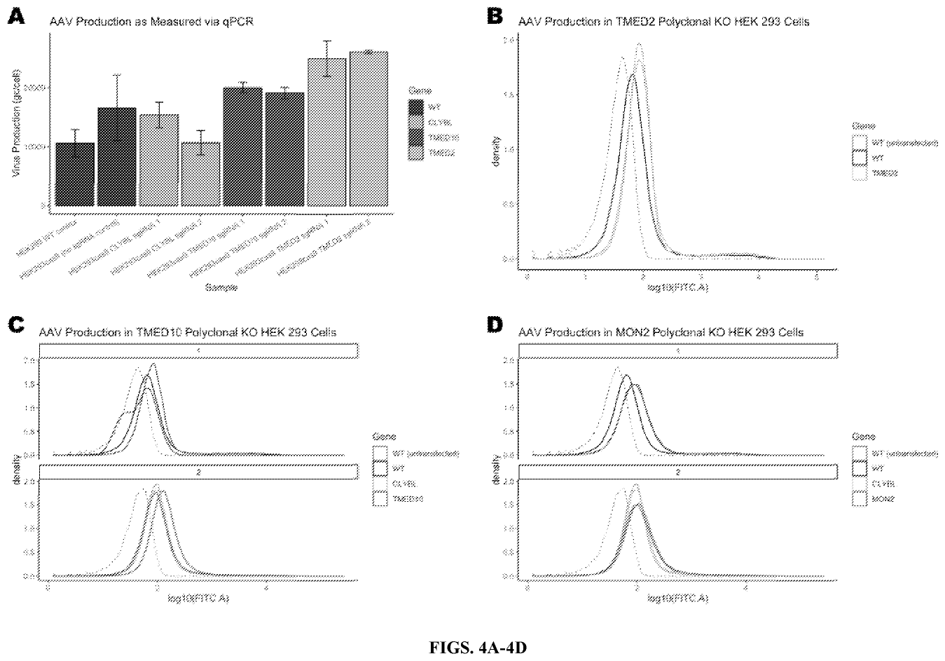

To further examine the role of the heparan sulfate biosynthesis pathway on AAV vector production, polyclonal B3GAT3, EXT1, and B4GALT7 knockout lines were generated from wildtype HEK 293 cells. These were three of the top hits from the heparan sulfate biosynthesis pathway. As a control, wildtype HEK 293 cells expressing cas9 or polyclonal CLYBL knockout cells were used. Polyclonal B3GAT3 knockout cells and polyclonal EXT1 knockout cells underwent the AAV triple transfection followed by either qPCR or FACS. Both B3GAT3 knockout and EXT1 knockout reduced AAV production compared to control cells (FIGS. 3A, 3C, 3D). In addition, polyclonal B4GALT7 cell lines were generated from wildtype HEK 293. These cells underwent the AAV triple transfection followed by FACS analysis. These preliminary results suggest that B4GALT7 knockout reduces AAV expression compared to the control CLYBL knockout cells (FIG. 3B). Increasing the expression of these genes is expected to have the opposite effect on AAV production.

Next, one-by-one validation of the top gene hits was also performed to demonstrate that generation of cell lines with single gene knockouts, selected from the screen results, can provide cell lines with altered AAV production capacity. For this, polyclonal knockout lines were generated for the top three hits predicted to increase AAV production upon knockout (MON2, TMED10, TMED2). As a control, wildtype HEK 293 expressing cas9 or polyclonal CLYBL knockout cells were used. Polyclonal TMED2 and TMED10 knockout cell lines underwent AAV triple transfection followed by qPCR analysis. Both

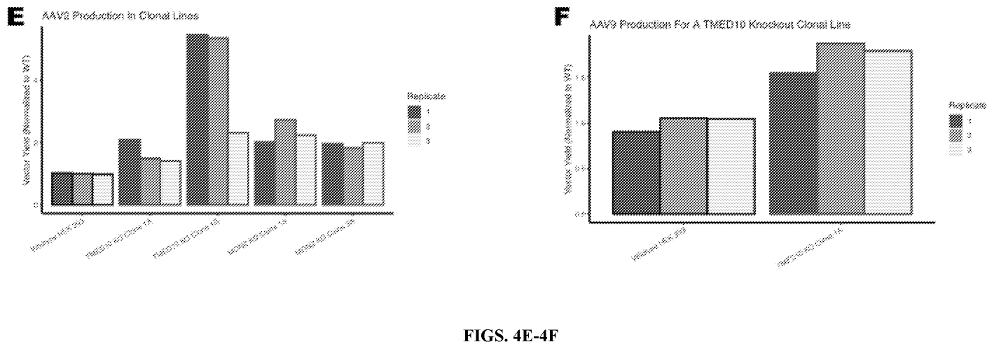

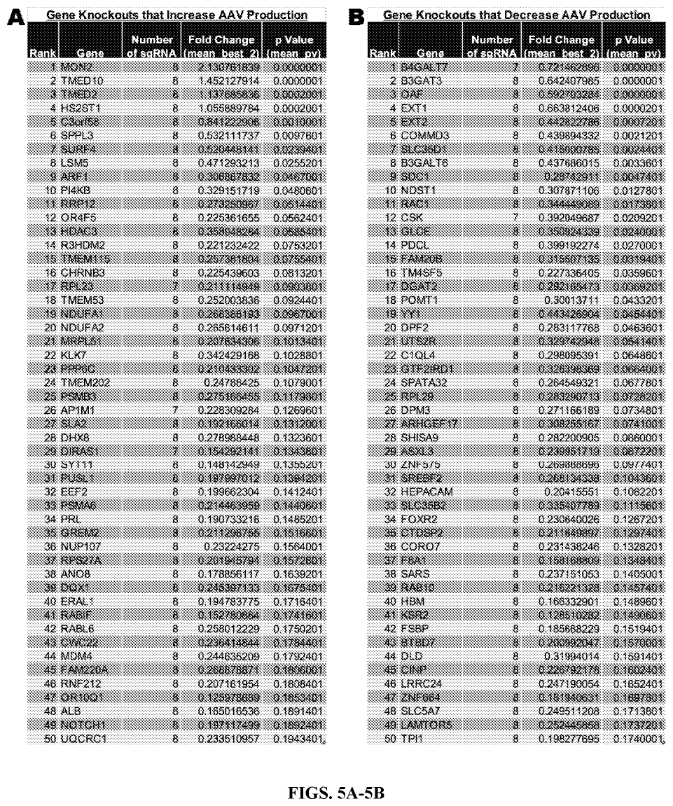

TMED2 knockout and TMED10 knockout increased AAV production compared to the controls (FIG. 4A). Polyclonal TMED2, TMED10, and MON2 knockout cells underwent AAV triple transfection followed by FACS 48 hours post-transfection. TMED2 knockout increased AAV production compared to wildtype cells (FIG. 4B). For the polyclonal TMED10 knockout cells, one of the two sgRNAs tested showed an increase in AAV production compared to either wildtype cells or CLYBL knockout cells (FIG. 4C). Polyclonal MON2 knockout cells led to an increase in in AAV production compared to control cells (FIG. 4D). Clonal TMED10 knockout lines and clonal MON2 knockout lines were used for AAV2 production, with three replicates per line. The TMED10 and MON2 knockout clonal lines all had increased vector yields compared to the wildtype HEK 293 cells (FIG. 4E). A clonal TMED10 knockout line showed increased AAV9 vector production compared to wildtype HEK 293 cells (FIG. 4F). This suggests that the top hits from the screen (FIGS. 5A, 5B) can increase AAV vector production for multiple AAV serotypes.

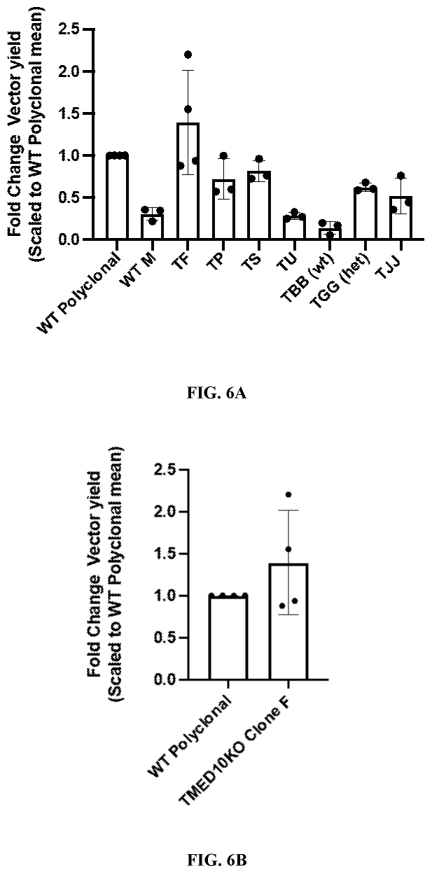

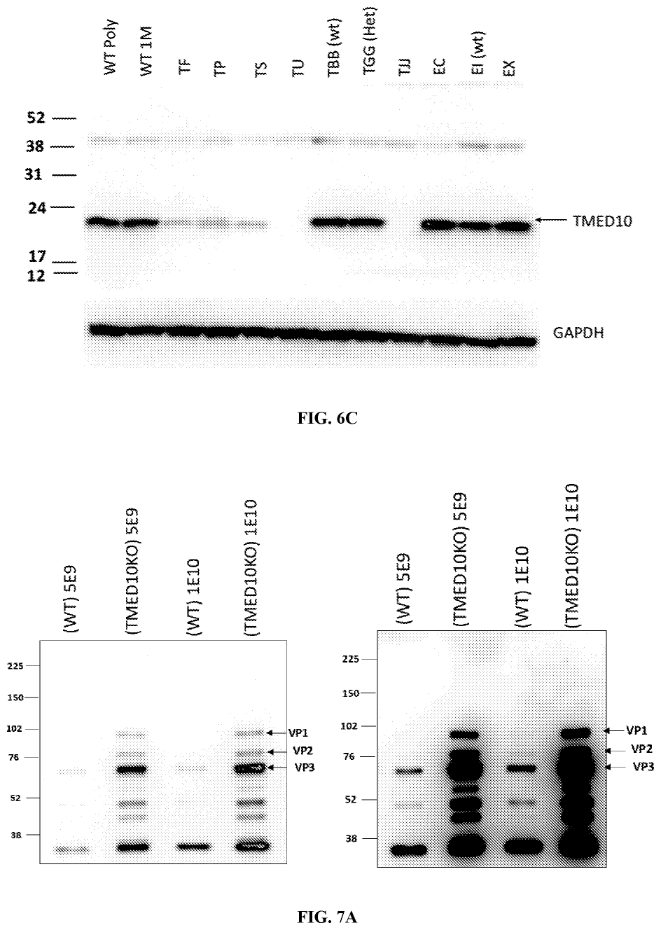

Additional clonal TMED10 knockout lines were generated and used for AAV2 production (FIGS. 6A-6C). In these experiments, AAV virus was produced by triple plasmid transfection at small scale (12-well format). Following DNase treatment and lysis, qPCR was performed to quantify the vector yield. The vector yield was normalized for cell count and transfection efficiency. Fold change was calculated by setting the yield from wild-type HEK293 to 1. Clone F was selected for further analysis because it showed 1.5 times more vector yield than wild-type HEK293 cells (FIG. 6B).

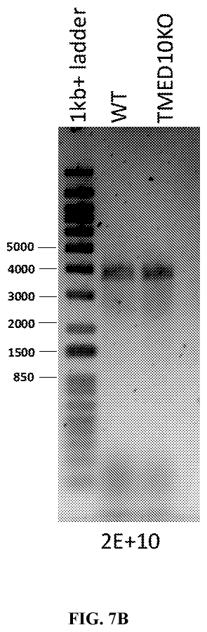

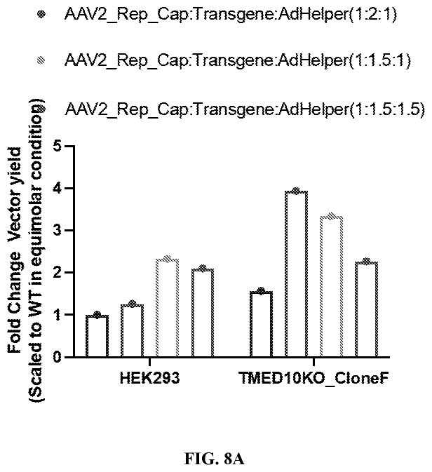

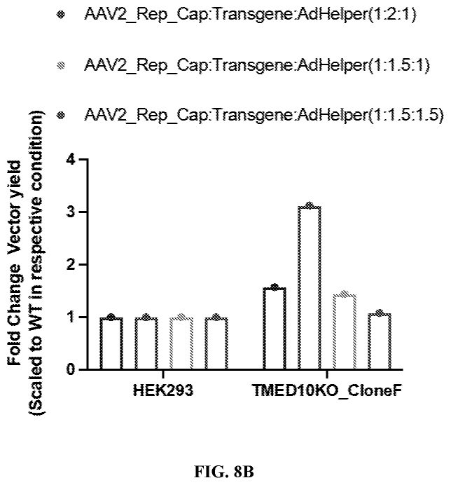

Next, AAV2 production was scaled up to a medium scale and the virus purified by Iodixanol. Quality control was carried out using western blot against VP proteins and alkaline gel electrophoresis for genome integrity (FIGS. 7A-7B). These data indicated that more capsids were empty in the TMED10 knockout AAV preparation than in the wild-type HEK293 AAV preparation. It was hypothesized that this might be due to TMED10 knockout cells producing more VP proteins as compared to wild-type HEK293 cells, even though they were each transfected with same amount. To test this, the molar ratio in which the three plasmid transfections were done was changed from the equimolar ratio used in standard conditions. Three non-equimolar different ratios were tested: AAV2_Rep_Cap: Transgene: AdHelper (1:1.5:1), AAV2_Rep_Cap: Transgene: AdHelper (1:2:1), and AAV2_Rep_Cap: Transgene: AdHelper (1:1.5:1.5). P2-SpCas9 was used as the transgene. At a ratio of 1:2:1 (Rep_Cap: Transgene: AdHelper), TMED10 knockout cells showed three times more vector yield as compared to wildtype HEK293 cells (FIGS. 8A-8B).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A genetically engineered producer cell line in which the expression of at least one of TMED10, MON2, TMED2, HS2ST1, C3orf58, SPPL3, SURF4, LSM5, ARF1, and PI4 KB is reduced as compared to a control cell line, and/or in which the expression of at least one of B4GALT7, B3GAT3, OAF, EXT2, COMMD3, SLC35D1, B3GALT6, SDC1, NDST1, RAC1, CSK, GLCE, PDCL, FAM20B, TM4SF5, DGAT2, POMT1, YY1, and DPF2 is increased as compared to a control cell line.

2. The genetically engineered producer cell line of claim 1, wherein the producer cell line is a human embryonic kidney 293T (HEK293T) cell line.

3. The genetically engineered producer cell line of claim 1 or 2, wherein the expression of at least one of TMED10, MON2, TMED2, HS2ST1, C3orf58, SPPL3, SURF4, LSM5, ARF1, and PI4 KB is reduced as compared to a control cell line.

4. The genetically engineered producer cell line of any one of claims 1-3, wherein the expression of at least one of TMED10, MON2, TMED2, HS2ST1, C3orf58, SPPL3, SURF4, LSM5, ARF1, and PI4 KB is reduced using CRISPR genome editing, a double stranded RNA (dsRNA), a small interfering RNA (siRNA), a small hairpin RNA (shRNA),a micro RNA or an antisense RNA.

5. The genetically engineered producer cell line of any one of claims 1-3, wherein at least one of TMED10, MON2, TMED2, HS2ST1, C3orf58, SPPL3, SURF4, LSM5, ARF1, and PI4 KB is knocked out.

6. The genetically engineered producer cell line of any one of claims 1-5, wherein the expression of one or more of the additional genes listed in FIGS. 5A and 5B is modulated in the producer cell line.

7. The genetically engineered producer cell line of claim 3 or 4, in which the expression of TMED10, MON2, and/or TMED2 are reduced as compared to a control cell line.

8. The genetically engineered producer cell line of claim 7, which does not express TMED10, MON2, and/or TMED2.

9. The genetically engineered producer cell line of any one of claims 1-8, comprising a viral vector.

10. A method for producing adeno-associated viral (AAV) particles comprising culturing a producer cell line in which the expression of at least one of TMED10, MON2, TMED2, HS2ST1, C3orf58, SPPL3, SURF4, LSM5, ARF1, and PI4 KB is reduced as compared to a control cell line, and/or in which the expression of at least one of B4GALT7, B3GAT3, OAF, EXT2, COMMD3, SLC35D1, B3GALT6, SDC1, NDST1, RAC1, CSK, GLCE, PDCL, FAM20B, TM4SF5, DGAT2, POMT1, YY1, and DPF2 is increased as compared to a control cell line, under condition allowing production and/or secretion of the AAV particles.

11. The method of claim 10, wherein the producer cell line is a human embryonic kidney 293T (HEK293T) cell line.

12. The method of claim 10 or 11, wherein the expression of at least one of TMED10, MON2, TMED2, HS2ST1, C3orf58, SPPL3, SURF4, LSM5, ARF1, and PI4 KB is reduced as compared to a control cell line.

13. The method of any one of claims 10-12, wherein the expression of at least one of TMED10, MON2, TMED2, HS2ST1, C3orf58, SPPL3, SURF4, LSM5, ARF1, and PI4 KB is reduced using CRISPR genome editing, a double stranded RNA (dsRNA), a small interfering RNA (siRNA), a small hairpin RNA (shRNA),a micro RNA or an antisense RNA.

14. The method of any one of claims 10-12, wherein at least one of TMED10, MON2, TMED2, HS2ST1, C3orf58, SPPL3, SURF4, LSM5, ARF1, and PI4 KB is knocked out.

15. The method of any one of claims 1-5, wherein the expression of one or more of the additional genes listed in FIGS. 5A and 5B is modulated in the producer cell line.

16. The method of any one of claims 10-15, wherein the titer of AAV particles is increased at least 1.5-fold compared with the titer of AAV particles produced by a control method.

17. The method of any one of claims 10-16, wherein the producer cell line is stably or transiently transfected with a first heterologous nucleic acid sequence, and optionally stably or transiently transfected with a second heterologous nucleic acid sequence.

18. The method of claim 17, wherein the first heterologous nucleic acid sequence encodes a therapeutic protein or polynucleotide sequence and the optional second heterologous nucleic acid sequence encodes a therapeutic protein or polynucleotide sequence.

19. The method of claim 18, wherein the therapeutic protein or polynucleotide sequence encoded by the first heterologous nucleic acid sequence and the therapeutic protein or polynucleotide sequence encoded by the optional second heterologous nucleic acid sequence are the same or are different.

20. The method of any one of claims 17-19, wherein the therapeutic polynucleotide sequence is an inhibitory nucleic acid sequence, and wherein the inhibitory nucleic acid sequence comprises a small or short hairpin (sh)RNA, microRNA (miRNA), small or short interfering (si) RNA, trans-splicing RNA, or antisense RNA.

21. The method of any one of claims 17-20, wherein the first heterologous nucleic acid sequence is comprised within a first viral vector, and the optional second heterologous nucleic acid sequence is comprised within a second viral vector.

22. The method of claim 21, wherein the first and/or second viral vector comprises an AAV vector genome.

23. The method of claim 22, wherein the AAV vector genome(s) comprises one or two AAV ITRs that flank the 5′ and/or 3′ ends of the heterologous nucleic acid sequence.

24. The method of any one of claims 10-23, wherein the producer cell line further comprises AAV rep and/or cap encoding sequences.

25. The method of claim 24, wherein the producer cell line is either transiently or stably transfected with a plasmid that comprises AAV rep and/or cap encoding sequences.

26. The method of any one of claims 10-25, wherein the producer cell line further comprises AAV helper function encoding sequences.

27. The method of any one of claims 10-26, wherein the method comprises transfecting the cells with a first plasmid encoding AAV rep and/or cap, a second plasmid encoding AAV helper function sequences, and a third plasmid encoding a transgene.

28. The method of claim 27, wherein the first plasmid, the second plasmid, and the third plasmid are transfected at a ratio of 1 to 1 to 1.

29. The method of claim 27, wherein the first plasmid, the second plasmid, and the third plasmid are transfected at a ratio of 1 to 1 to 1.5, 1 to 1 to 2, or 1 to 1.5 to 1.5.

30. The method of claim 29, wherein the first plasmid, the second plasmid, and the third plasmid are transfected at a ratio of 1 to 1 to 2.

31. The method of any one of claims 10-30, wherein the titer of AAV particles is increased at least 3-fold compared with the titer of AAV particles produced by a control method.

32. The method of any one of claims 26-31, wherein the AAV helper function sequences are a sequence encoding adenovirus E4, a sequence encoding adenovirus E2A, and/or a sequence encoding adenovirus VA RNA.

33. The method of any one of claims 17-32, wherein the producer cell line produces AAV particles having packaged therein the heterologous nucleic acid sequence(s).

34. The method of any one of claims 10-33, wherein the AAV particles are produced in greater amounts by the producer cell line than a control cell line.

35. The method of any one of claims 10-34, further comprising isolating or purifying the AAV particles from the cell culture.

36. A population of adeno-associated viral (AAV) particles obtainable from the producer cell line of any one of claims 1 to 9, or by the method of any one of claims 10 to 29.

38. The population of AAV particles of claim 36, wherein the AAV particles are of an AAV1, AAV2, and/or AAV9 serotype.

39. A pharmaceutical composition comprising the AAV particles of claim 36 or 37 and a pharmaceutically acceptable carrier.

40. A method of treating a disease in a mammal comprising administering the pharmaceutical composition of claim 39 to the mammal.
